(19) United States
(12) Patent Application Publication
(10) Pub. No.: US 2022/0382029 A1
(43) Pub. Date: Dec. 1, 2022

(54) DENTAL MICROSCOPE

(71) Applicant: Tsung-Lung Hsieh, Kaohsiung City (TW)

(72) Inventor: Tsung-Lung Hsieh, Kaohsiung City (TW)

(21) Appl. No.: 17/454,334

(22) Filed: Nov. 10, 2021

(30) Foreign Application Priority Data
May 25, 2021 (TW) 110205993

(51) Int. Cl. G02B 21/00; G02B 21/36; G02B 21/02; A61B 90/25

(52) U.S. Cl. CPC G02B 21/0004; G02B 21/368; G02B 21/025; A61B 90/25

(57) ABSTRACT

A dental microscope includes a microscope unit, an adjustable support arm, and a display unit. The microscope unit includes a body part, at least one eyepiece that is disposed on the body part, and an objective lens disposed on a bottom end of the body part. The adjustable support arm has a first end connected to the microscope unit and a second end opposite to the first end. The adjustable support arm is adjustable in a segment-by-segment manner to move the second end relative to the first end. The display unit is connected to the second end of the adjustable support arm and is in signal communication with the microscope unit for displaying a captured image obtained by the microscope unit.

### CROSS-REFERENCE TO RELATED APPLICATIONS

This application claims priority of Taiwanese Patent Application No. 110205993, filed on May 25, 2021.

### FIELD

The disclosure relates to a dental microscope, more particularly to a dental microscope equipped with a display unit.

### BACKGROUND

With reference to FIG. 1, a conventional dental microscope system to be used in cooperation with a dental chair 11 for a patient A1 to sit in is illustrated. The conventional dental microscope system includes a microscope 12 set above the dental chair 11, and a display 13 located on a lateral side of the dental chair 11, spaced apart from the microscope 12 and in signal communication with the microscope 12. The microscope 12 is operable by a dentist (A2) to move above the face of the patient (A1), so as to produce an image of the oral cavity of the patient (A1) and to take pictures or record video of the oral cavity. The display 13 is a screen provided to display in real time the video of the oral cavity recorded by the microscope 12 for viewing by a dental assistant (A3) standing by the dental chair 11. During diagnosis and treatment, the dental assistant (A3) provides assistance, such as positioning suction devices, passing instruments to the dentist (A2), and so on. When providing assistance, the dental assistant (A3) must avoid obstructing the view of the microscope 12 under the operation of the dentist (A2). Therefore, the dental assistant (A3) must always pay attention to the field of view of the dentist (A2) by viewing the screen 13 to provide accurate assistance and avoid injuring the patient (A1).

However, the positioning of the display 13 is awkward for the dental assistant A3, who will need to divide his/her attention between the oral cavity of the patient (A1) to the display 13. Under these circumstances, due to the awkward positioning of the display 13, the dental assistant (A3) will need to turn his/her neck in order to view the display 13; at this moment, the dental assistant (A3) cannot provide timely assistance, and the repetitive neck movements may lead to fatigue and soreness.

### SUMMARY

Therefore, the object of the disclosure is to provide a dental microscope equipped with a display unit that is more convenient for use in diagnosis and treatment and that can further improve work efficiency.

According to the disclosure, the dental microscope includes a microscope unit, an adjustable support arm, and a display unit. The microscope unit includes a body part that extends along a vertical direction, at least one eyepiece that is disposed on the body part, and an objective lens. The objective lens and the body part are arranged along the vertical direction. The objective lens is disposed on a bottom end of the body part. The objective lens produces an image. The at least one eyepiece magnifies the image for viewing therethrough. The image produced by the objective lens is captured by the microscope unit to obtain a captured image. The microscope unit and the adjustable support arm are arranged along a horizontal direction perpendicular to the vertical direction. The adjustable support arm has a first end connected to the microscope unit and a second end opposite to the first end. The adjustable support arm is adjustable in a segment-by-segment manner to move the second end relative to the first end. The display unit is connected to the second end of the adjustable support arm and is in signal communication with the microscope unit for displaying the captured image obtained by the microscope unit.

### BRIEF DESCRIPTION OF THE DRAWINGS

Other features and advantages of the disclosure will become apparent in the following detailed description of the embodiments with reference to the accompanying drawings, of which:

FIG. 1 is a schematic diagram illustrating a conventional dental microscope system;

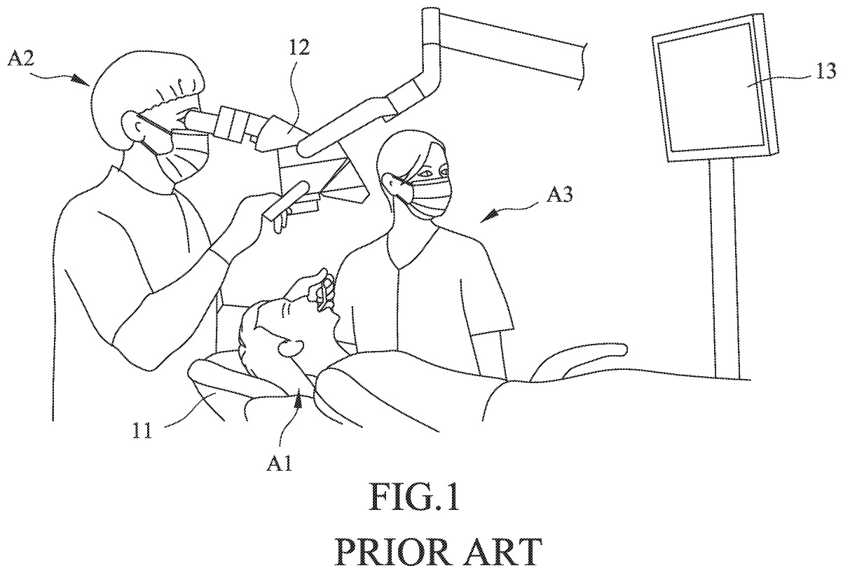

FIG. 2 is a perspective view illustrating an embodiment of a dental microscope according to the disclosure;

FIG. 3 is a fragmentary perspective view illustrating the connection between an adjustable support arm and a second ball-and-socket connector of the embodiment;

FIG. 4 is a fragmentary perspective view illustrating the connection between the adjustable support arm and a first ball-and-socket connector of the embodiment;

FIG. 5 is a fragmentary perspective view illustrating a variation of the adjustable support arm of the embodiment; and

FIG. 6 is a schematic diagram illustrating the embodiment in use.

### DETAILED DESCRIPTION

Before the present invention is described in greater detail, it should be noted that where considered appropriate, reference numerals or terminal portions of reference numerals have been repeated among the figures to indicate corresponding or analogous elements, which may optionally have similar characteristics.

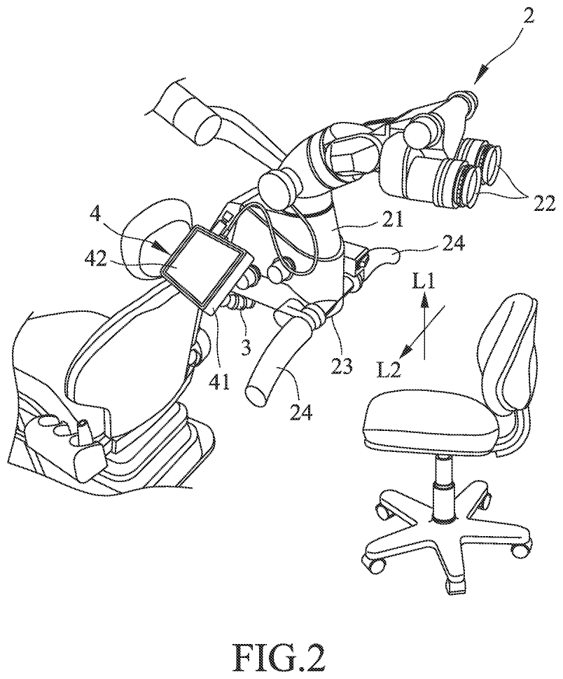

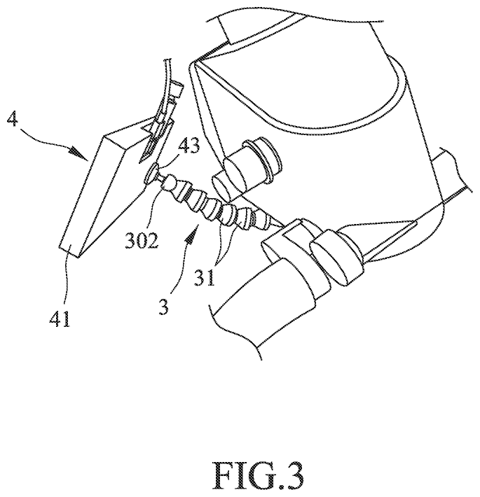

As shown in FIGS. 2 to 4, the embodiment of a dental microscope according to the present disclosure includes a microscope unit 2, an adjustable support arm 3 disposed on the microscope unit 2, and a display unit 4 connected to the adjustable support arm 3.

In this embodiment, the microscope unit 2 includes a body part 21 extending along a vertical direction (L1), two eyepieces 22 disposed on the body part 21 and spaced apart from each other along a horizontal direction (L2) perpendicular to the vertical direction (L1), and an objective lens 23. The objective lens 23 and the body part 21 are arranged along the vertical direction (L1), and the objective lens 23 is disposed on a bottom end of the body part 21. The microscope unit 2 further includes two handlebars 24 arranged on the body part 21 along the horizontal direction (L2), and a first ball-and-socket connector 25 projecting horizontally from the body part 21. The objective lens 23 produces an image, and the image is magnified by the eyepieces 22 and can be viewed through the eyepieces 22 as a magnified image.

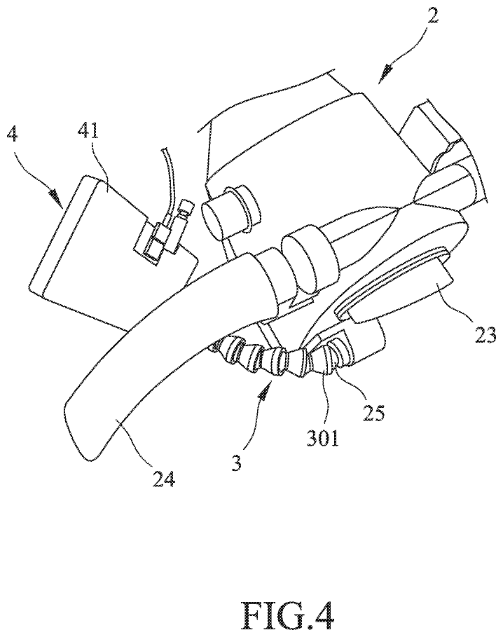

The adjustable support arm 3 is elongated, and is connected to a lateral side of the microscope unit 2 (i.e., the microscope unit 2 and the adjustable support arm 3 are arranged along the horizontal direction (L2)). The adjustable support arm 3 includes a plurality of sequentially arranged articulated segments 31 operable to change the shape of the adjustable support arm 3 and to allow the adjustable support arm 3 to keep the shape thus changed. A terminal one of the articulated segments is connected to the first ball-and-socket connector 25 of the microscope unit 2 and referred to as a first end 301 of the adjustable support arm 3. The other terminal one of the articulated segments 31, opposite to the first end 301, is connected to the display unit 4 and referred to as a second end 302 of the adjustable support arm 3. The first end 301 includes one of a ball head and a socket base coupled to each other, while the first ball-and-socket connector 25 includes the other one of the ball head and the socket base. The first end 301 and the first ball-and-socket connector 25 cooperate to form a ball-and-socket joint. In this embodiment as shown in FIG. 4, the first end 301 includes a socket base, and the first ball-and-socket connector 25 includes a ball head. Each articulated segment 31 is rotatable relative to adjacent one(s) of the articulated segments 31 within a limited range in all directions so that the adjustable support arm 3 can be adjusted in a segment-by-segment manner to move the second end 302 relative to the first end 301. In this embodiment, any two adjacent articulated segments 31 are also connected in a manner of a ball-and-socket joint. In addition, by rotating the first end 301 relative to the first ball-and-socket connector 25, the adjustable support arm 3 can swivel relative to the first ball-and-socket connector 25, which further improves the operational flexibility of the adjustable support arm 3.

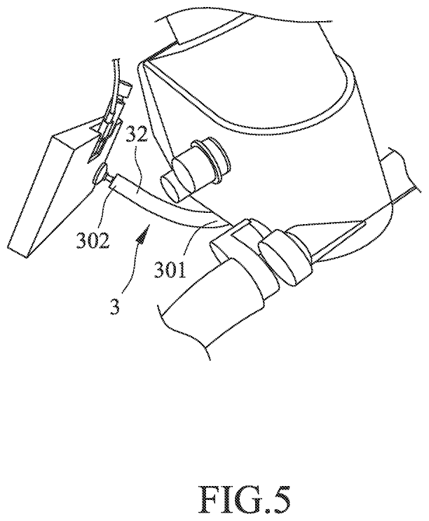

As shown in FIG. 5, a variation of the adjustable support arm 3 is a flexible gooseneck tube that includes a tube body 32. The tube body 32 includes an inner spring, and an outer wire wound around the inner spring and compressed into gaps between coils of the inner spring. The tube body 32 has opposite ends that are connected respectively to the microscope unit 2 and the display unit 4 and that serve respectively as the first end 301 and the second end 302 of the adjustable support arm 3. The tube body 32 can be adjusted segment-by-segment, by virtue of the structures of the coils of the inner spring and the outer wire, to move the second end 302 relative to the first end 301 and to allow the second end 302 to stay in position after being moved. Referring to FIGS. 2 to 4, the display unit 4 is in signal communication with the microscope unit for synchronously displaying a captured image produced by the microscope unit 2. In one embodiment, the captured image is related to the image produced by the objective lens 23, and is obtained by an image sensor included in the microscope unit 2. The captured image may be a dynamic image, or may be plural in number and form a video. The display unit 4 includes a housing 41, a screen 42 embedded in the housing 41 for displaying image content (e.g., the captured image), and a second ball-and-socket connector 43 projecting from the housing 41 and connected to the second end 302 of the adjustable support arm 3. Similar to the first end 301 and the first ball-and-socket connector 25, the second end 302 includes one of a ball head and a socket base coupled to each other, while the second ball-and-socket connector 43 includes the other one of the ball head and the socket base. The second end 302 and the second ball-and-socket connector 43 cooperate to form a ball-and-socket joint. In this embodiment, the second end 302 includes a socket base, and the second ball-and-socket connector 43 includes a ball head. In particular, through the adjustable support arm 3 which interconnects the microscope unit 2 and the display unit 4, the display unit 4 can be moved along with the microscope unit 2 as the microscope unit 2 moves. By virtue of the structure of the adjustable support arm 3 that allows segment-by-segment adjustment, the display unit 4 can be moved relative to the microscope unit 2 with high operational flexibility. In addition, through the mechanism of ball-and-socket joint formed by the second ball-and-socket connector 43 and the second end 302, the display unit 4 can be rotated relative to the adjustable support arm 3, which further improves its operational capability.

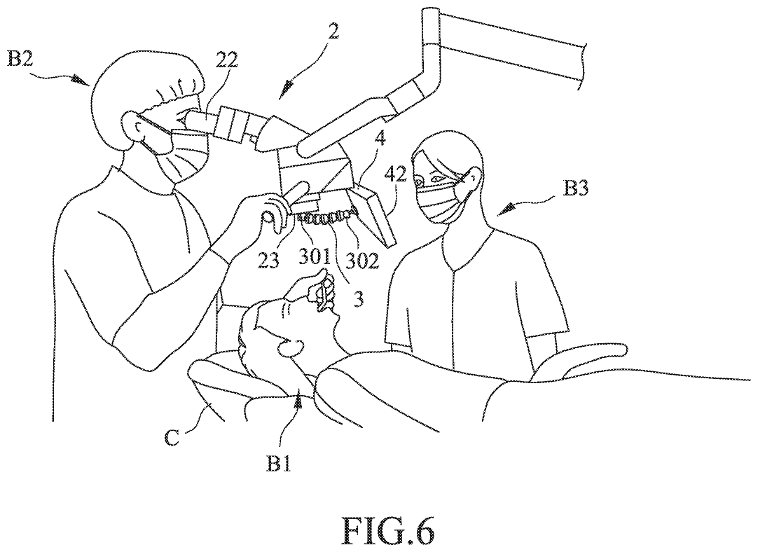

Referring to FIGS. 2 and 6, during the process of diagnosis and treatment, this dental microscope is set above a dental chair (C) that a patient (B1) sits in, and a dentist (B2) controls one of the handlebars 24 to adjust the microscope unit 2 to an appropriate position. Through the objective lens 23, an image of the oral cavity of the patient (B1) is produced. The dentist (B2) can view the image that is magnified by the eyepieces 22 through the eyepieces 22 to carry out diagnosis and treatment. Usually, a dental assistant (B3) stands next to the dental chair (C), and provides relevant support and assistance to the dentist (B2) so as to facilitate the diagnosis and treatment. The dental assistant (B3) can adjust the relative position of the display unit 4 and the microscope unit 2 and place the display unit 4 near the patient's oral cavity so that both the screen 42 of the display unit 4 and the patient's oral cavity are in the field of view of the dental assistant (B3). Therefore, the dental assistant (B3) can observe the patient's oral condition in real-time through viewing the captured image displayed on the screen 42 without turning his/her neck. In this way, the dental assistant (B3) is able to provide accurate assistance in a timely manner during the process so as to improve the efficiency of diagnosis and treatment, and can also avoid accidental injury to the patient (B1). In addition, due to the advantageous positioning of the display unit 4 relative to the patient's oral cavity, the dental assistant (B3) is less prone to fatigue or neck soreness, even during extended treatments, which can help avoid occupational injuries.

In summary, for the dental microscope according to the present disclosure, through adjusting the adjustable support arm 3 segment-by-segment to change the relative position of its first end 301 and second end 302, the display unit 4 that is connected with the second end 302 can be moved relative to the microscope unit 2. The display unit 4 and patient's oral cavity can be both in the dental assistant's field of view, thereby facilitating the viewing of the captured image, improving work efficiency, and even avoiding occupational injuries so that the purpose of the present disclosure can be achieved.

In the description above, for the purposes of explanation, numerous specific details have been set forth in order to provide a thorough understanding of the embodiments. It will be apparent, however, to one skilled in the art, that one or more other embodiments may be practiced without some of these specific details. It should also be appreciated that reference throughout this specification to “one embodiment,” “an embodiment,” an embodiment with an indication of an ordinal number and so forth means that a particular feature, structure, or characteristic may be included in the practice of the disclosure. It should be further appreciated that in the description, various features are sometimes grouped together in a single embodiment, figure, or description thereof for the purpose of streamlining the disclosure and aiding in the understanding of various inventive aspects, and that one or more features or specific details from one embodiment may be practiced together with one or more features or specific details from another embodiment, where appropriate, in the practice of the disclosure.

While the disclosure has been described in connection with what is considered the exemplary embodiment, it is understood that this disclosure is not limited to the disclosed embodiments but is intended to cover various arrangements included within the spirit and scope of the broadest interpretation so as to encompass all such modifications and equivalent arrangements.

What is claimed is:

1. A dental microscope comprising:
a microscope unit including a body part that extends along a vertical direction, at least one eyepiece that is disposed on said body part, and an objective lens, said objective lens and said body part being arranged along the vertical direction, said objective lens being disposed on a bottom end of said body part, said objective lens producing an image, said at least one eyepiece magnifying the image for viewing therethrough, the image produced by said objective lens being captured by said microscope unit to obtain a captured image;
an adjustable support arm, said microscope unit and said adjustable support arm being arranged along a horizontal direction perpendicular to said vertical direction, said adjustable support arm having a first end connected to said microscope unit and a second end opposite to said first end, said adjustable support arm being adjustable in a segment-by-segment manner to move said second end relative to said first end; and
a display unit connected to said second end of said adjustable support arm and being in signal communication with said microscope unit for displaying the captured image obtained by said microscope unit.

2. The dental microscope equipped as claimed in claim 1, wherein said adjustable support arm includes a plurality of sequentially arranged articulated segments, each of said articulated segments being rotatable relative to an adjacent one of said articulated segments, a terminal one of said articulated segments that is connected to said microscope unit serving as said first end of said adjustable support arm, the other terminal one of said articulated segments that is connected to said display unit serving as said second end of said adjustable support arm.

3. The dental microscope as claimed in claim 1, wherein said adjustable support arm is a gooseneck tube that has a tube body, said tube body having opposite ends that serve as said first end and said second end of said adjustable support arm, respectively.

4. The dental microscope as claimed in claim 1, wherein said microscope unit further includes a first ball-and-socket connector projecting horizontally from said body part and connected to said first end of said adjustable support arm.

5. The dental microscope as claimed in claim 1, wherein said display unit includes a housing, a screen embedded in said housing, and a second ball-and-socket connector projecting from said housing and connected to said second end of said adjustable support arm.
